(19) United States
(12) Patent Application Publication
(10) Pub. No.: US 2013/0109972 A1
(43) Pub. Date: May 2, 2013

(54) ULTRASONIC DIAGNOSTIC APPARATUS

(71) Applicant: GE Medical Systems Global Technology Compan, Waukesha, WI (US)

(72) Inventor: Lei LIU, Tokyo (JP)

(73) Assignee: GE MEDICAL SYSTEMS GLOBAL TECHNOLOGY COMPANY, LLC, Waukesha, WI (US)

(21) Appl. No.: 13/661,475

(22) Filed: Oct. 26, 2012

(30) Foreign Application Priority Data
Oct. 28, 2011 (JP) 2011-236959

(51) Int. Cl. A61B 8/06

(52) U.S. Cl. 600/454

(57) ABSTRACT

An ultrasonic diagnostic apparatus is provided. The ultrasonic diagnostic apparatus includes a B-flow data generator configured to generate B-flow data based on echo signals obtained by performing transmission/reception of ultrasound on a subject given vibrations, and a displayer configured to display a B-flow image based on the B-flow data.

### CROSS REFERENCE TO RELATED APPLICATIONS

This application claims the benefit of Japanese Patent Application No. 2011-236959 filed Oct. 28, 2011, which is hereby incorporated by reference in its entirety.

### BACKGROUND OF THE INVENTION

The present invention relates to an ultrasonic diagnostic apparatus that displays a B-flow image.

There has been known an ultrasonic diagnostic apparatus which displays an ultrasonic image generated based on echo signals obtained by transmitting ultrasound to a subject. There has been disclosed a B-flow image capable of bringing a blood flow dynamic state relative to a stationary biological tissue into imaging as an ultrasonic image (refer to, for example, Richard Y. Chiao, Larry Y. Mo et al., “B-Mode Blood Flow (B-FLOW) Imaging”, Ultrasonics Symposium, 2000 IEEE, USA, IEEE, 2000, vol. 2, PP. 1469-1472, and Nishioka Makiko, “B-flow based Flow Imaging inclusive of 3D Method”, Clinical Image, Medical View Co., Ltd, May 2008, vol. 24, No. 5, p. 627-630). It has been known that a B-flow image in which each moving portion is displayed in color is also included in such a B-flow image in addition to a monochrome B-flow image (refer to, for example, Hamazaki Naoki et al., “the usefulness of B-FLOW COLOR for the subpleural lesions”, Japanese Journal of Clinical Radiology, 2007, vol. 52, No. 1, p. 119-128).

Incidentally, “A New Marker for Diagnosis of Thyroid Papillary Cancer” by Luca Brunese et al., J Ultrasound Med, USA, American Institute of Ultrasound in Medicine, 2008, vol. 27, p. 1187-1194 discloses that since each of microcalcified parts that occur in a breast tissue is displayed in high brightness in a monochrome B-flow image, the B-flow image is suitable for observation of the microcalcified parts.

### BRIEF DESCRIPTION OF THE INVENTION

In a monochrome B-flow image, the brightness of each substance moving starting with a blood flow is displayed high. Accordingly, the reason why each of microcalcified parts is displayed in brightness higher than a peripheral tissue is considered to be that the microcalcified part vibrates due to the sound pressure of transmitted ultrasound. The microcalcs may be displayed in color even in the B-flow color image.

Thus, the utility of the B-flow image has been recognized in the observation of microstructures such as the microcalcified parts or the like. With the above foregoing in view, studies have been performed on the fact that the B-flow image makes it easier to recognize the microstructures.

The embodiments described herein include an ultrasonic diagnostic apparatus equipped with a B-flow data generator which generates B-flow data, based on echo signals obtained by performing transmission/reception of ultrasound on a subject given vibrations, and a displayer which displays a B-flow image based on the B-flow data thereon.

As each microstructure in a subject given vibrations vibrates upon the transmission/reception of ultrasound, a B-flow image generated based on echo signals obtained from the subject is higher than conventional in terms of detectability of the microstructure. It is thus possible to display the B-flow image that makes it easier to recognize the microstructures.

### BRIEF DESCRIPTION OF THE DRAWINGS

FIG. 1 is a block diagram showing one example of a schematic configuration of an ultrasonic diagnostic apparatus according to a first embodiment.

FIG. 2 is a diagram showing a B-flow image illustrative of an embodiment displayed on a displayer.

FIG. 3 is a diagram showing a conventional B-flow image displayed on the displayer.

FIG. 4 is a block diagram showing one example of a schematic configuration of an ultrasonic diagnostic apparatus according to a second embodiment.

FIG. 5 is a diagram showing an ultrasonic diagnostic apparatus and a placement table on which a subject is placed.

### DETAILED DESCRIPTION OF THE INVENTION

Exemplary embodiments will hereinafter be described.

### First Embodiment

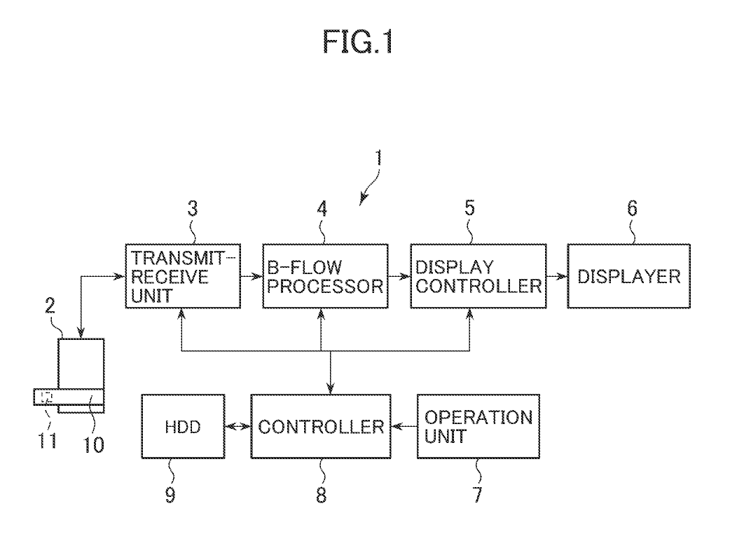

A first embodiment will first be described based on FIGS. 1 through 4. An ultrasonic diagnostic apparatus 1 shown in FIG. 1 is equipped with an ultrasonic probe 2, a transmit-receive unit 3, a B-flow processor 4, a display controller 5, a displayer 6, an operation unit 7, a controller 8 and an HDD (Hard Disk Drive) 9.

The ultrasonic probe 2 transmits ultrasound from a plurality of ultrasonic transducers (not shown) to a subject. The ultrasonic probe 2 performs ultrasound scanning sequentially every sound ray to transmit ultrasound. The ultrasonic probe 2 receives echo signals of the ultrasound at the ultrasonic transducers. The ultrasonic probe 2 is one example illustrative of an embodiment of an ultrasonic probe.

A transducing unit 11 is mounted to the ultrasonic probe 2 through a bracket 10. The transducing unit 11 is, for example, a vibration motor provided in the bracket 10. The transducing unit 11 vibrates at a specific frequency f. For example, the frequency f may be 25 Hz or 50 Hz. It is however needless to say that the frequency described herein is a mere illustration. The transducing unit 11 is one example illustrative of an embodiment of a transducing unit.

The transmit-receive unit 3 supplies an electric signal for transmitting ultrasound from the ultrasonic probe 2 under a predetermined scan condition to the ultrasonic probe 2 based on a control signal outputted from the controller 8. The transmit-receive unit 3 performs signal processing such as A/D conversion, phasing-adding processing, etc. on each echo signal received by the ultrasonic probe 2.

The B-flow processor 4 performs B-flow processing on data about the echo signals outputted from the transmit-receive unit 3 to generate B-flow data. The B-flow processor 4 is one example illustrative of an embodiment of a B-flow data generator.

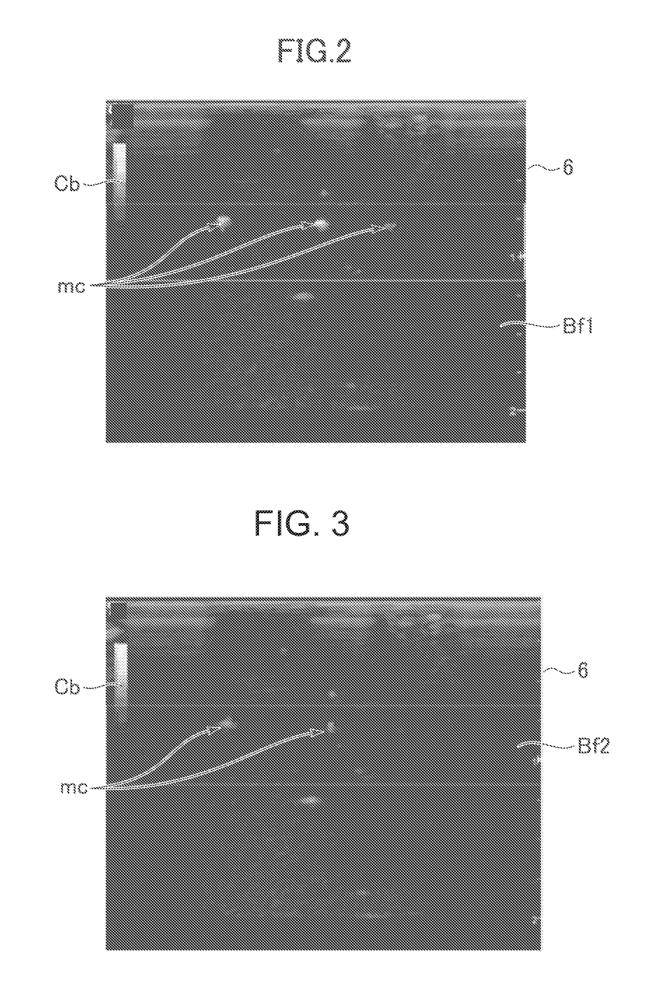

The display controller 5 scan-converts the B-flow data acquired by the B-flow processor 4 by means of a scan converter to generate B-flow image data. The display controller 5 also displays a B-flow image based on the B-flow image data on the displayer 6. The B-flow image is a monochrome image in which the brightness of a movable body is higher than the brightness of a stationary body, and a color image (B-flow color image) having a hue corresponding to the velocity of the movable body or the direction of its movement. B-flow color images are shown in FIGS. 2 and 3 to be described later.

The displayer 6 is comprised of, for example, an LCD (Liquid Crystal Display) or a CRT (Cathode Ray Tube). The displayer 6 is one example illustrative of an embodiment of a displayer.

The operation unit 7 is made up of a keyboard and a pointing device (not shown) or the like for inputting instructions and information by an operator.

Although not shown in the drawing in particular, the controller 8 is comprised of a CPU (central Processing Unit). The controller 8 reads a program stored in the HDD 9 to execute functions at the respective parts of the ultrasonic diagnostic apparatus 1.

The operation of the ultrasonic diagnostic apparatus 1 according to the present embodiment will now be explained. The operator performs transmission/reception of ultrasound through the ultrasonic probe 2 in a state in which the ultrasonic probe 2 is being in contact with the surface of a target region in the subject. The region target for the transmission/reception of the ultrasound is, for example, a breast. The ultrasound transmission/reception is carried out in a state in which the transducing unit 11 is vibrating. The controller 8 outputs a control signal to the transmit-receive unit 3 in such a manner that the transmission/reception of the ultrasound by the ultrasonic probe 2 is carried out with scan parameters suitable for the generation of B-flow data.

The echo signals received by the ultrasonic probe 2 are signal-processed by the transmit-receive unit 3. The B-flow processor 4 generates B-flow data, based on the data inputted from the transmit-receive unit 3. Then, the display controller 5 generates B-flow image data, based on the B-flow data and causes the displayer 6 to display a B-flow image Bf1 as shown in FIG. 2.

The B-flow image Bf1 is a color B-flow image. A color bar Cb made up of a hue corresponding to the velocity of movement of the movable body or the direction of its movement is displayed on the left side of the B-flow image Bf1 . Symbols mc respectively indicate microcalcified parts. The microcalcified parts mc are displayed in color.

In the B-flow image Bf1, microstructures such as microcalcified parts in a biological tissue, etc. are displayed in color. Here, a conventional B-flow image Bf2 formed where a target region is not given vibrations, is shown in FIG. 3. The B-flow image Bf2 is also a B-flow color image.

The B-flow image Bf1 shown in FIG. 2 is an image higher than the B-flow image Bf2 shown in FIG. 3 in terms of detectability of each microcalcified part mc. Specifically, such microcalcified parts mc as unconfirmable in the B-flow image Bf2 can be confirmed in the B-flow image Bf1, and microcalcified parts mc confirmable even in the B-flow image Bf2 are displayed in more emphasized form. The reasons therefor will be explained below.

At the transmission/reception of the ultrasound by the ultrasonic probe 2, the transducing unit 11 vibrates at a specific frequency f. Thus, the ultrasonic probe 2 vibrates and its vibrations are propagated into a biological tissue of a breast being a target region.

The vibrations of the transducing unit 11 are propagated through the bracket 10 and the ultrasonic probe 2 to propagate into the biological tissue of the breast. In the process of propagation of such vibrations, a plurality of frequencies equivalent to n (where n is a natural number) times the frequency f are generated. Thus, the vibrations propagated into the biological tissue include vibrations of frequencies 2 nf, 3 nf, 4 nf, etc. in addition to the frequency f.

Here, each microcalcified part in the biological tissue vibrates by the ultrasound transmitted from the ultrasonic probe 2. Of the vibrations propagated into the biological tissue by the vibrations of the transducing unit 11, the vibrations of frequency equal to the natural frequency of the microcalcified part vibrating by the ultrasound cause the microcalcified part to vibrate in resonance. Thus, the B-flow image Bf1 high in detectability of each microcalcified part mc can be obtained. The microcalcified part mc can easily be recognized in the B-flow image Bf1.

### Second Embodiment

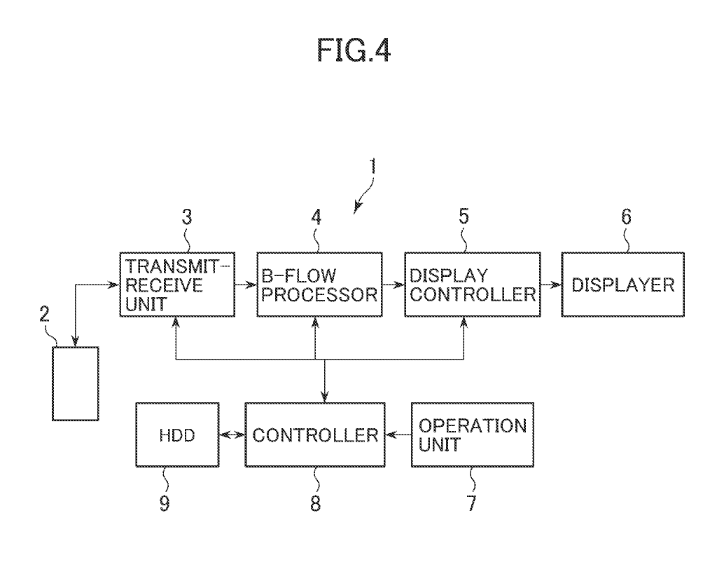

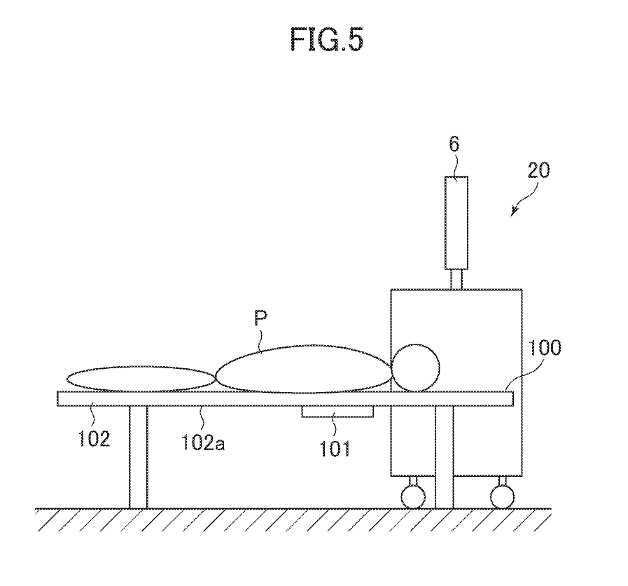

A second embodiment will next be explained based on FIGS. 4 and 5. The same reference numerals are respectively attached to the same components as those in the first embodiment, and their description will be omitted.

In the ultrasonic diagnostic apparatus 20 shown in FIG. 4, the ultrasonic probe 2 is not provided with the transducing unit 11. The second embodiment is identical to the first embodiment in other configuration.

In the present embodiment, as shown in FIG. 5, a placement table 100 on which a subject P is placed is provided with a transducing unit 101 instead of the transducing unit 11. The transducing unit 101 is one example illustrative of an embodiment of a transducing unit. The placement table 100 is one example illustrative of an embodiment of a placement table.

Although the transducing unit 101 is provided on the back surface 102a at the top plate 102 of the placement table 100 in FIG. 5, the transducing unit 101 may be embedded in the top plate 102. The transducing unit 101 is a vibration motor that vibrates at a specific frequency f as with the transducing unit 11.

Incidentally, the ultrasonic probe 2 in the ultrasonic diagnostic apparatus 20 is not shown in FIG. 5.

Even in the present embodiment, the transmission/reception of ultrasound is carried out at the ultrasonic probe 2 while the transducing unit 101 is being vibrated, so that a B-flow image is displayed on the displayer 6. The transducing unit 101 vibrates at the specific frequency f. The top plate 102 vibrates with the vibrations of the transducing unit 101, which vibrations are propagated into a biological tissue of a breast being a region target for the transmission/reception of the ultrasound. As with the first embodiment, a plurality of frequencies equivalent to n times the frequency f are generated in the process of the propagation of the vibrations. Of the vibrations of frequencies f, 2 nf, 3 nf, 4 nf, etc. propagated into the biological tissue of the chest region, the vibration of any frequency will cause each microcalcified part to vibrate in resonance. It is thus possible to easily recognize each microcalcified part in the B-flow image in a manner similar to the first embodiment.

Although exemplary embodiments are described above, it is needless to say that the present invention may be modified in various ways within the scope that does not change the spirit of the invention. In the first embodiment, for example, the transducing unit 11 may be provided within the ultrasonic probe 2.

1. An ultrasonic diagnostic apparatus comprising:
a B-flow data generator configured to generate B-flow data based on echo signals obtained by performing transmission/reception of ultrasound on a subject given vibrations; and
a displayer configured to display a B-flow image based on the B-flow data.

2. The ultrasonic diagnostic apparatus according to claim 1, wherein the vibrations are given by a transducing unit included in an ultrasonic probe configured to perform the transmission/reception of ultrasound on the subject.

3. The ultrasonic diagnostic apparatus according to claim 1, wherein the vibrations are given by a transducing unit included with a placement table on which the subject is placed.

4. The ultrasonic diagnostic apparatus according to claim 1, wherein a plurality of vibrations having frequencies equivalent to n times the frequency of the vibrations given to the subject cause an object to be observed to vibrate in resonance, wherein n is a natural number.

5. The ultrasonic diagnostic apparatus according to claim 2, wherein a plurality of vibrations having frequencies equivalent to n times the frequency of the vibrations given to the subject cause an object to be observed to vibrate in resonance, wherein n is a natural number.

6. The ultrasonic diagnostic apparatus according to claim 3, wherein a plurality of vibrations having frequencies equivalent to n times the frequency of the vibrations given to the subject cause an object to be observed to vibrate in resonance, wherein n is a natural number.

7. The ultrasonic diagnostic apparatus according to claim 4, wherein the the object is also vibrated by the ultrasound transmitted to the subject.

8. The ultrasonic diagnostic apparatus according to claim 5, wherein the the object is also vibrated by the ultrasound transmitted to the subject.

9. The ultrasonic diagnostic apparatus according to claim 6, wherein the the object is also vibrated by the ultrasound transmitted to the subject.

10. The ultrasonic diagnostic apparatus according to claim 7, wherein the object to be observed is a microstructure.

11. The ultrasonic diagnostic apparatus according to claim 10, wherein the microstructure is a microcalcified part of a biological tissue.

12. The ultrasonic diagnostic apparatus according to claim 1, further comprising an ultrasonic probe configured to perform transmission/reception of ultrasound with scan parameters to generate the B-flow data.

13. The ultrasonic diagnostic apparatus according to claim 2, further comprising an ultrasonic probe configured to perform transmission/reception of ultrasound with scan parameters to generate the B-flow data.

14. The ultrasonic diagnostic apparatus according to claim 3, further comprising an ultrasonic probe configured to perform transmission/reception of ultrasound with scan parameters to generate the B-flow data.

15. The ultrasonic diagnostic apparatus according to claim 4, further comprising an ultrasonic probe configured to perform transmission/reception of ultrasound with scan parameters to generate the B-flow data.

16. The ultrasonic diagnostic apparatus according to claim 7, further comprising an ultrasonic probe configured to perform transmission/reception of ultrasound with scan parameters to generate the B-flow data.

17. The ultrasonic diagnostic apparatus according to claim 1, wherein a B-flow color image is included in the B-flow image.

18. A method for imaging a subject, said method comprising:
transmitting vibrations to the subject;
performing transmission/reception of ultrasound on the subject to obtain echo signals;
generating, using a B-flow data generator, B-flow data based on the obtained echo signals; and
displaying on a displayer a B-flow image based on the generated B-flow data.

19. A method in accordance with claim 18, wherein transmitting vibrations to the subject comprises transmitting vibrations to the subject using a transducing unit of an ultrasound probe.

20. An ultrasonic system comprising:
an ultrasonic diagnostic apparatus comprising:
a B-flow data generator configured to generate B-flow data based on echo signals obtained by performing transmission/reception of ultrasound on a subject experiencing vibrations; and
a displayer configured to display a B-flow image based on the B-flow data; and
an ultrasound probe coupled to said ultrasonic diagnostic apparatus and configure to perform the transmission/reception of ultrasound on the subject, said ultrasound probe comprising a transducing unit configured to supply the vibrations to the subject.
